(19) United States
(12) Patent Application Publication
(10) Pub. No.: US 2022/0218596 A1
(43) Pub. Date: Jul. 14, 2022

(54) COMPOSITIONS FOR BEAUTY PRODUCTS AND METHODS OF MAKING SAME

(71) Applicant: Karen Dupiche, East Orange, NJ (US)

(72) Inventor: Karen Dupiche, East Orange, NJ (US)

(21) Appl. No.: 17/573,824

(22) Filed: Jan. 12, 2022

Related U.S. Application Data
(60) Provisional application No. 63/136,281, filed on Jan. 12, 2021.

(51) Int. Cl. A61K 8/9794; A61K 8/9717; A61Q 1/02; A61K 8/04

(52) U.S. Cl. CPC A61K 8/9794; A61K 8/042; A61Q 1/02; A61K 8/9717

(57) ABSTRACT

A composition includes Aloe vera gel of 5% to 95% by weight of the composition; and sea moss of 5% to 95% by weight of the composition. A method includes the steps of obtaining a quantity of Aloe Vera plant; obtaining a quantity of sea moss; rinsing the sea moss with water for a period of 30 seconds to two minutes, wherein a temperature of the water is 95° F. to 115° F.; storing the sea moss in a refrigeration unit for a period of 4 hours to 36 hours; mixing the sea moss; storing the mixed sea moss in a refrigeration unit for a period of 4 hours to 36 hours; obtaining an interior gel of the Aloe vera plant; and mixing the sea moss with the Aloe vera gel.

### CROSS-REFERENCE TO RELATED APPLICATION

This application is a Section 111(a) application relating to and claiming the benefit of commonly-owned, co-pending U.S. Nonprovisional Patent Application Ser. No. 63/136,281, filed Jan. 12, 2021, entitled “COMPOSITIONS FOR BEAUTY PRODUCTS AND METHODS OF MAKING SAME,” the contents of which are incorporated herein by reference in its entirety.

### FIELD OF THE INVENTION

The present invention relates to beauty products and, more particularly, compositions used in the manufacturing of beauty products and methods for making same.

### BACKGROUND OF THE INVENTION

There are a wide variety of beauty products for skin care. What is needed is a natural beauty care product that promotes healthier and younger looking skin, particularly for the skin and area under a person's eyes.

### SUMMARY

In some embodiments, a composition includes Aloe vera gel, wherein the Aloe vera gel is 5% to 95% by weight of the composition; and sea moss, wherein the sea moss is 5% to 95% by weight of the composition. In some embodiments, the sea moss is Chondrus crispus. In some embodiments, the composition is configured for us a concealer primer. In some embodiments, the composition is configured for use as a topical preparation. In some embodiments, the composition is configured for use as a skin preparation. In some embodiments, the composition is configured for use as a body preparation. In some embodiments, the composition is configured for use as a cream. In some embodiments, the composition is configured for use as a lotion.

In some embodiments, a method includes the steps of: obtaining a quantity of Aloe Vera plant; obtaining a quantity of sea moss; rinsing the sea moss with water for a period of 30 seconds to two minutes, wherein a temperature of the water is 95° F. to 115° F.; storing the sea moss in a refrigeration unit for a period of 4 hours to 36 hours; mixing the sea moss; storing the mixed sea moss in a refrigeration unit for a period of 4 hours to 36 hours; obtaining an interior gel of the Aloe vera plant; and mixing the sea moss with the Aloe vera gel. In some embodiments, the method further includes the step of storing the mixed sea moss and the Aloe vera gel in a refrigeration unit for a period. In some embodiments, the sea moss of the storing the sea moss step is stored in the refrigeration unit at a temperature of 35° F. to 45° F. In some embodiments, the mixed sea moss of the storing the mixed sea moss step is stored in the refrigeration unit at a temperature of 35° F. to 45° F. In some embodiments, the sea moss of the storing the sea moss step is stored in the refrigeration unit for 24 hours. In some embodiments, the mixed sea moss of the storing the mixed sea moss step is stored in the refrigeration unit for 24 hours. In some embodiments, the mixing the sea moss step includes mixing the sea moss in a blender. In some embodiments, a weight of the sea moss is 10 grams to 50 grams. In some embodiments, a weight of the Aloe vera gel is 10 grams to 50 grams.

In some embodiments, the mixing the mixed sea moss with the Aloe vera gel step includes mixing the mixed sea moss and the Aloe vera gel for 30 seconds to two minutes. In some embodiments, the mixing the mixed sea moss with the Aloe vera gel step includes mixing the mixed sea moss and the Aloe vera gel in a blender. In some embodiments, the mixing the mixed sea moss with the Aloe vera gel step includes mixing the mixed sea moss and the Aloe vera gel manually.

### DETAILED DESCRIPTION

In an embodiment, a composition for a beauty product includes Aloe vera gel and sea moss. In an embodiment, the Aloe vera gel is obtained from an Aloe vera plant, a succulent plant species of the genus Aloe. In an embodiment, the Aloe vera is configured to evoke positive effects on skin disorders, such as psoriasis, acne and rashes. In an embodiment, the sea moss is “Chondrus crispus,” Irish moss or carrageen moss, which is a species of red algae obtained for sea/ocean areas. In an embodiment, the sea moss 15 is configured to act as an immunity booster.

In an embodiment, the composition includes 5% to 95% Aloe vera gel by weight of the composition. In another embodiment, the composition includes 5% to 90% Aloe vera gel by weight of the composition. In another embodiment, the composition includes 5% to 85% Aloe vera gel by weight of the composition. In another embodiment, the composition includes 5% to 80% Aloe vera gel by weight of the composition. In another embodiment, the composition includes 5% to 75% Aloe vera gel by weight of the composition. In another embodiment, the composition includes 5% to 70% Aloe vera gel by weight of the composition. In another embodiment, the composition includes 5% to 65% Aloe vera gel by weight of the composition. In another embodiment, the composition includes 5% to 60% Aloe vera gel by weight of the composition. In another embodiment, the composition includes 5% to 55% Aloe vera gel by weight of the composition. In another embodiment, the composition includes 5% to 50% Aloe vera gel by weight of the composition. In another embodiment, the composition includes 5% to 45% Aloe vera gel by weight of the composition. In another embodiment, the composition includes 5% to 40% Aloe vera gel by weight of the composition. In another embodiment, the composition includes 5% to 35% Aloe vera gel by weight of the composition. In another embodiment, the composition includes 5% to 30% Aloe vera gel by weight of the composition. In another embodiment, the composition includes 5% to 25% Aloe vera gel by weight of the composition. In another embodiment, the composition includes 5% to 20% Aloe vera gel by weight of the composition. In another embodiment, the composition includes 5% to 15% Aloe vera gel by weight of the composition. In another embodiment, the composition includes 5% to 10% Aloe vera gel by weight of the composition.

In an embodiment, the composition includes 10% to 95% Aloe vera gel by weight of the composition. In another embodiment, the composition includes 10% to 90% Aloe vera gel by weight of the composition. In another embodiment, the composition includes 10% to 85% Aloe vera gel by weight of the composition. In another embodiment, the composition includes 10% to 80% Aloe vera gel by weight of the composition. In another embodiment, the composition includes 10% to 75% Aloe vera gel by weight of the composition. In another embodiment, the composition includes 10% to 70% Aloe vera gel by weight of the composition. In another embodiment, the composition includes 10% to 65% Aloe vera gel by weight of the composition. In another embodiment, the composition includes 10% to 60% Aloe vera gel by weight of the composition. In another embodiment, the composition includes 10% to 55% Aloe vera gel by weight of the composition. In another embodiment, the composition includes 10% to 50% Aloe vera gel by weight of the composition. In another embodiment, the composition includes 10% to 45% Aloe vera gel by weight of the composition. In another embodiment, the composition includes 10% to 40% Aloe vera gel by weight of the composition. In another embodiment, the composition includes 10% to 35% Aloe vera gel by weight of the composition. In another embodiment, the composition includes 10% to 30% Aloe vera gel by weight of the composition. In another embodiment, the composition includes 10% 10 to 25% Aloe vera gel by weight of the composition. In another embodiment, the composition includes 10% to 20% Aloe vera gel by weight of the composition. In another embodiment, the composition includes 10% to 15% Aloe vera gel by weight of the composition.

In an embodiment, the composition includes 15% to 95% Aloe vera gel by weight of the composition. In another embodiment, the composition includes 15% to 90% Aloe vera gel by weight of the composition. In another embodiment, the composition includes 15% to 85% Aloe vera gel by weight of the composition. In another embodiment, the composition includes 15% to 80% Aloe vera gel by weight of the composition. In another embodiment, the composition includes 15% to 75% Aloe vera gel by weight of the composition. In another embodiment, the composition includes 15% to 70% Aloe vera gel by weight of the composition. In another embodiment, the composition includes 15% to 65% Aloe vera gel by weight of the composition. In another embodiment, the composition includes 15% to 60% Aloe vera gel by weight of the composition. In another embodiment, the composition includes 15% to 55% Aloe vera gel by weight of the composition. In another embodiment, the composition includes 15% to 50% Aloe vera gel by weight of the composition. In another embodiment, the composition includes 15% to 45% Aloe vera gel by weight of the composition. In another embodiment, the composition includes 15% to 40% Aloe vera gel by weight of the composition. In another embodiment, the composition includes 15% to 35% Aloe vera gel by weight of the composition. In another embodiment, the composition includes 15% to 30% Aloe vera gel by weight of the composition. In another embodiment, the composition includes 15% to 25% Aloe vera gel by weight of the composition. In another embodiment, the composition includes 15% to 20% Aloe vera gel by weight of the composition.

In an embodiment, the composition includes 20% to 95% Aloe vera gel by weight of the composition. In another embodiment, the composition includes 20% to 90% Aloe vera gel by weight of the composition. In another embodiment, the composition includes 20% to 85% Aloe vera gel by weight of the composition. In another embodiment, the composition includes 20% to 80% Aloe vera gel by weight of the composition. In another embodiment, the composition includes 20% to 75% Aloe vera gel by weight of the composition. In another embodiment, the composition includes 20% to 70% Aloe vera gel by weight of the composition. In another embodiment, the composition includes 20% to 65% Aloe vera gel by weight of the composition. In another embodiment, the composition includes 20% to 60% Aloe vera gel by weight of the composition. In another embodiment, the composition includes 20% to 55% Aloe vera gel by weight of the composition. In another embodiment, the composition includes 20% to 50% Aloe vera gel by weight of the composition. In another embodiment, the composition includes 20% to 45% Aloe vera gel by weight of the composition. In another embodiment, the composition includes 20% to 40% Aloe vera gel by weight of the composition. In another embodiment, the composition includes 20% to 35% Aloe vera gel by weight of the composition. In another embodiment, the composition includes 20% to 30% Aloe vera gel by weight of the composition. In another embodiment, the composition includes 20% to 25% Aloe vera gel by weight of the composition.

In an embodiment, the composition includes 25% to 95% Aloe vera gel by weight of the composition. In another embodiment, the composition includes 25% to 90% Aloe vera gel by weight of the composition. In another embodiment, the composition includes 25% to 85% Aloe vera gel by weight of the composition. In another embodiment, the composition includes 25% to 80% Aloe vera gel by weight of the composition. In another embodiment, the composition includes 25% to 75% Aloe vera gel by weight of the composition. In another embodiment, the composition includes 25% to 70% Aloe vera gel by weight of the composition. In another embodiment, the composition includes 25% to 65% Aloe vera gel by weight of the composition. In another embodiment, the composition includes 25% to 60% Aloe vera gel by weight of the composition. In another embodiment, the composition includes 25% to 55% Aloe vera gel by weight of the composition. In another embodiment, the composition includes 25% to 50% Aloe vera gel by weight of the composition. In another embodiment, the composition includes 25% to 45% Aloe vera gel by weight of the composition. In another embodiment, the composition includes 25% to 40% Aloe vera gel by weight of the composition. In another embodiment, the composition includes 25% to 35% Aloe vera gel by weight of the composition. In another embodiment, the composition includes 25% to 30% Aloe vera gel by weight of the composition.

In an embodiment, the composition includes 30% to 95% Aloe vera gel by weight of the composition. In another embodiment, the composition includes 30% to 90% Aloe vera gel by weight of the composition. In another embodiment, the composition includes 30% to 85% Aloe vera gel by weight of the composition. In another embodiment, the composition includes 30% to 80% Aloe vera gel by weight of the composition. In another embodiment, the composition includes 30% to 75% Aloe vera gel by weight of the composition. In another embodiment, the composition includes 30% to 70% Aloe vera gel by weight of the composition. In another embodiment, the composition includes 30% to 65% Aloe vera gel by weight of the composition. In another embodiment, the composition includes 30% to 60% Aloe vera gel by weight of the composition. In another embodiment, the composition includes 30% to 55% Aloe vera gel by weight of the composition. In another embodiment, the composition includes 30% to 50% Aloe vera gel by weight of the composition. In another embodiment, the composition includes 30% to 45% Aloe vera gel by weight of the composition. In another embodiment, the composition includes 30% to 40% Aloe vera gel by weight of the composition. In another embodiment, the composition includes 30% to 35% Aloe vera gel by weight of the composition.

In an embodiment, the composition includes 35% to 95% Aloe vera gel by weight of the composition. In another embodiment, the composition includes 35% to 90% Aloe vera gel by weight of the composition. In another embodiment, the composition includes 35% to 85% Aloe vera gel by weight of the composition. In another embodiment, the composition includes 35% to 80% Aloe vera gel by weight of the composition. In another embodiment, the composition includes 35% to 75% Aloe vera gel by weight of the composition. In another embodiment, the composition includes 35% to 70% Aloe vera gel by weight of the composition. In another embodiment, the composition includes 35% to 65% Aloe vera gel by weight of the composition. In another embodiment, the composition includes 35% to 60% Aloe vera gel by weight of the composition. In another embodiment, the composition includes 35% to 55% Aloe vera gel by weight of the composition. In another embodiment, the composition includes 35% to 50% Aloe vera gel by weight of the composition. In another embodiment, the composition includes 35% 10 to 45% Aloe vera gel by weight of the composition. In another embodiment, the composition includes 35% to 40% Aloe vera gel by weight of the composition.

In an embodiment, the composition includes 40% to 95% Aloe vera gel by weight of the composition. In another embodiment, the composition includes 40% to 90% Aloe vera gel by weight of the composition. In another embodiment, the composition includes 40% to 85% Aloe vera gel by weight of the composition. In another embodiment, the composition includes 40% to 80% Aloe vera gel by weight of the composition. In another embodiment, the composition includes 40% to 75% Aloe vera gel by weight of the composition. In another embodiment, the composition includes 40% to 70% Aloe vera gel by weight of the composition. In another embodiment, the composition includes 40% to 65% Aloe vera gel by weight of the composition. In another embodiment, the composition includes 40% to 60% Aloe vera gel by weight of the composition. In another embodiment, the composition includes 40% to 55% Aloe vera gel by weight of the composition. In another embodiment, the composition includes 40% to 50% Aloe vera gel by weight of the composition. In another embodiment, the composition includes 40% to 45% Aloe vera gel by weight of the composition.

In an embodiment, the composition includes 45% to 95% Aloe vera gel by weight of the composition. In another embodiment, the composition includes 45% to 90% Aloe vera gel by weight of the composition. In another embodiment, the composition includes 45% to 85% Aloe vera gel by weight of the composition. In another embodiment, the composition includes 45% to 80% Aloe vera gel by weight of the composition. In another embodiment, the composition includes 45% to 75% Aloe vera gel by weight of the composition. In another embodiment, the composition includes 45% to 70% Aloe vera gel by weight of the composition. In another embodiment, the composition includes 45% to 65% Aloe vera gel by weight of the composition. In another embodiment, the composition includes 45% to 60% Aloe vera gel by weight of the composition. In another embodiment, the composition includes 45% to 55% Aloe vera gel by weight of the composition. In another embodiment, the composition includes 45% to 50% Aloe vera gel by weight of the composition.

In an embodiment, the composition includes 50% to 95% Aloe vera gel by weight of the composition. In another embodiment, the composition includes 50% to 90% Aloe vera gel by weight of the composition. In another embodiment, the composition includes 50% to 85% Aloe vera gel by weight of the composition. In another embodiment, the composition includes 50% to 80% Aloe vera gel by weight of the composition. In another embodiment, the composition includes 50% to 75% Aloe vera gel by weight of the composition. In another embodiment, the composition includes 50% to 70% Aloe vera gel by weight of the composition. In another embodiment, the composition includes 50% to 65% Aloe vera gel by weight of the composition. In another embodiment, the composition includes 50% to 60% Aloe vera gel by weight of the composition. In another embodiment, the composition includes 50% to 55% Aloe vera gel by weight of the composition.

In an embodiment, the composition includes 55% to 95% Aloe vera gel by weight of the composition. In another embodiment, the composition includes 55% to 90% Aloe vera gel by weight of the composition. In another embodiment, the composition includes 55% to 85% Aloe vera gel by weight of the composition. In another embodiment, the composition includes 55% to 80% Aloe vera gel by weight of the composition. In another embodiment, the composition includes 55% to 75% Aloe vera gel by weight of the composition. In another embodiment, the composition includes 55% to 70% Aloe vera gel by weight of the composition. In another embodiment, the composition includes 55% to 65% Aloe vera gel by weight of the composition. In another embodiment, the composition includes 55% to 60% Aloe vera gel by weight of the composition.

In an embodiment, the composition includes 60% to 95% Aloe vera gel by weight of the composition. In another embodiment, the composition includes 60% to 90% Aloe vera gel by weight of the composition. In another embodiment, the composition includes 60% to 85% Aloe vera gel by weight of the composition. In another embodiment, the composition includes 60% to 80% Aloe vera gel by weight of the composition. In another embodiment, the composition includes 60% to 75% Aloe vera gel by weight of the composition. In another embodiment, the composition includes 60% to 70% Aloe vera gel by weight of the composition. In another embodiment, the composition includes 60% to 65% Aloe vera gel by weight of the composition.

In an embodiment, the composition includes 65% to 95% Aloe vera gel by weight of the composition. In another embodiment, the composition includes 65% to 90% Aloe vera gel by weight of the composition. In another embodiment, the composition includes 65% to 85% Aloe vera gel by weight of the composition. In another embodiment, the composition includes 65% to 80% Aloe vera gel by weight of the composition. In another embodiment, the composition includes 65% to 75% Aloe vera gel by weight of the composition. In another embodiment, the composition includes 65% to 70% Aloe vera gel by weight of the composition.

In an embodiment, the composition includes 70% to 95% Aloe vera gel by weight of the composition. In another embodiment, the composition includes 70% to 90% Aloe vera gel by weight of the composition. In another embodiment, the composition includes 70% to 85% Aloe vera gel by weight of the composition. In another embodiment, the composition includes 70% to 80% Aloe vera gel by weight of the composition. In another embodiment, the composition includes 70% to 75% Aloe vera gel by weight of the composition.

In an embodiment, the composition includes 75% to 95% Aloe vera gel by weight of the composition. In another embodiment, the composition includes 75% to 90% Aloe vera gel by weight of the composition. In another embodiment, the composition includes 75% to 85% Aloe vera gel by weight of the composition. In another embodiment, the composition includes 75% to 80% Aloe vera gel by weight of the composition.

In an embodiment, the composition includes 80% to 95% Aloe vera gel by weight of the composition. In another embodiment, the composition includes 80% to 90% Aloe vera gel by weight of the composition. In another embodiment, the composition includes 80% to 85% Aloe vera gel by weight of the composition.

In an embodiment, the composition includes 85% to 95% Aloe vera gel by weight of the composition. In another embodiment, the composition includes 85% to 90% Aloe vera gel by weight of the composition.

In an embodiment, the composition includes 90% to 95% Aloe vera gel by weight of the composition.

In an embodiment, the composition includes 5% Aloe vera gel by weight of the composition. In another embodiment, the composition includes 10% Aloe vera gel by weight of the composition. In another embodiment, the composition includes 15% Aloe vera gel by weight of the composition. In another embodiment, the composition includes 20% Aloe vera gel by weight of the composition. In another embodiment, the composition includes 25% Aloe vera gel by weight of the composition. In another embodiment, the composition includes 30% Aloe vera gel by weight of the composition. In another embodiment, the composition includes 35% Aloe vera gel by weight of the composition. In another embodiment, the composition includes 40% Aloe vera gel by weight of the composition. In another embodiment, the composition includes 45% Aloe vera gel by weight of the composition. In another embodiment, the composition includes 50% Aloe vera gel by weight of the composition. In another embodiment, the composition includes 55% Aloe vera gel by weight of the composition. In another embodiment, the composition includes 60% Aloe vera gel by weight of the composition. In another embodiment, the composition includes 65% Aloe vera gel by weight of the composition. In another embodiment, the composition includes 70% Aloe vera gel by weight of the composition. In another embodiment, the composition includes 75% Aloe vera gel by weight of the composition. In another embodiment, the composition includes 80% Aloe vera gel by weight of the composition. In another embodiment, the composition includes 85% Aloe vera gel by weight of the composition. In another embodiment, the composition includes 90% Aloe vera gel by weight of the composition. In another embodiment, the composition includes 95% Aloe vera gel by weight of the composition.

In an embodiment, the composition includes 5% to 95% sea moss by weight of the composition. In another embodiment, the composition includes 5% to 90% sea moss by weight of the composition. In another embodiment, the composition includes 5% to 85% sea moss by weight of the composition. In another embodiment, the composition includes 5% to 80% sea moss by weight of the composition. In another embodiment, the composition includes 5% to 75% sea moss by weight of the composition. In another embodiment, the composition includes 5% to 70% sea moss by weight of the composition. In another embodiment, the composition includes 5% to 65% sea moss by weight of the composition. In another embodiment, the composition includes 5% to 60% sea moss by weight of the composition. In another embodiment, the composition includes 5% to 55% sea moss by weight of the composition. In another embodiment, the composition includes 5% to 50% sea moss by weight of the composition. In another embodiment, the composition includes 5% to 45% sea moss by weight of the composition. In another embodiment, the composition includes 5% to 40% sea moss by weight of the composition. In another embodiment, the composition includes 5% to 35% sea moss by weight of the composition. In another embodiment, the composition includes 5% to 30% sea moss by weight of the composition. In another embodiment, the composition includes 5% to 25% sea moss by weight of the composition. In another embodiment, the composition includes 5% to 20% sea moss by weight of the composition. In another embodiment, the composition includes 5% to 15% sea moss by weight of the composition. In another embodiment, the composition includes 5% to 10% sea moss by weight of the composition.

In an embodiment, the composition includes 10% to 95% sea moss by weight of the composition. In another embodiment, the composition includes 10% to 90% sea moss by weight of the composition. In another embodiment, the composition includes 10% to 85% sea moss by weight of the composition. In another embodiment, the composition includes 10% to 80% sea moss by weight of the composition. In another embodiment, the composition includes 10% to 75% sea moss by weight of the composition. In another embodiment, the composition includes 10% to 70% sea moss by weight of the composition. In another embodiment, the composition includes 10% to 65% sea moss by weight of the composition. In another embodiment, the composition includes 10% to 60% sea moss by weight of the composition. In another embodiment, the composition includes 10% to 55% sea moss by weight of the composition. In another embodiment, the composition includes 10% to 50% sea moss by weight of the composition. In another embodiment, the composition includes 10% to 45% sea moss by weight of the composition. In another embodiment, the composition includes 10% to 40% sea moss by weight of the composition. In another embodiment, the composition includes 10% to 35% sea moss by weight of the composition. In another embodiment, the composition includes 10% to 30% sea moss by weight of the composition. In another embodiment, the composition includes 10% to 25% sea moss by weight of the composition. In another embodiment, the composition includes 10% to 20% sea moss by weight of the composition. In another embodiment, the composition includes 10% to 15% sea moss by weight of the composition.

In an embodiment, the composition includes 15% to 95% sea moss by weight of the composition. In another embodiment, the composition includes 15% to 90% sea moss by weight of the composition. In another embodiment, the composition includes 15% to 85% sea moss by weight of the composition. In another embodiment, the composition includes 15% to 80% sea moss by weight of the composition. In another embodiment, the composition includes 15% to 75% sea moss by weight of the composition. In another embodiment, the composition includes 15% to 70% sea moss by weight of the composition. In another embodiment, the composition includes 15% to 65% sea moss by weight of the composition. In another embodiment, the composition includes 15% to 60% sea moss by weight of the composition. In another embodiment, the composition includes 15% to 55% sea moss by weight of the composition. In another embodiment, the composition includes 15% to 50% sea moss by weight of the composition. In another embodiment, the composition includes 15% to 45% sea moss by weight of the composition. In another embodiment, the composition includes 15% to 40% sea moss by weight of the composition. In another embodiment, the composition includes 15% to 35% sea moss by weight of the composition. In another embodiment, the composition includes 15% to 30% sea moss by weight of the composition. In another embodiment, the composition includes 15% to 25% sea moss by weight of the composition. In another embodiment, the composition includes 15% to 20% sea moss by weight of the composition.

In an embodiment, the composition includes 20% to 95% sea moss by weight of the composition. In another embodiment, the composition includes 20% to 90% sea moss by weight of the composition. In another embodiment, the composition includes 20% to 85% sea moss by weight of the composition. In another embodiment, the composition includes 20% to 80% sea moss by weight of the composition. In another embodiment, the composition includes 20% to 75% sea moss by weight of the composition. In another embodiment, the composition includes 20% to 70% sea moss by weight of the composition. In another embodiment, the composition includes 20% to 65% sea moss by weight of the composition. In another embodiment, the composition includes 20% to 60% sea moss by weight of the composition. In another embodiment, the composition includes 20% to 55% sea moss by weight of the composition. In another embodiment, the composition includes 20% to 50% sea moss by weight of the composition. In another embodiment, the composition includes 20% to 45% sea moss by weight of the composition. In another embodiment, the composition includes 20% to 40% sea moss by weight of the composition. In another embodiment, the composition includes 20% to 35% sea moss by weight of the composition. In another embodiment, the composition includes 20% to 30% sea moss by weight of the composition. In another embodiment, the composition includes 20% to 25% sea moss by weight of the composition.

In an embodiment, the composition includes 25% to 95% sea moss by weight of the composition. In another embodiment, the composition includes 25% to 90% sea moss by weight of the composition. In another embodiment, the composition includes 25% to 85% sea moss by weight of the composition. In another embodiment, the composition includes 25% to 80% sea moss by weight of the composition. In another embodiment, the composition includes 25% to 75% sea moss by weight of the composition. In another embodiment, the composition includes 25% to 70% sea moss by weight of the composition. In another embodiment, the composition includes 25% to 65% sea moss by weight of the composition. In another embodiment, the composition includes 25% to 60% sea moss by weight of the composition. In another embodiment, the composition includes 25% to 55% sea moss by weight of the composition. In another embodiment, the composition includes 25% to 50% sea moss by weight of the composition. In another embodiment, the composition includes 25% to 45% sea moss by weight of the composition. In another embodiment, the composition includes 25% to 40% sea moss by weight of the composition. In another embodiment, the composition includes 25% to 35% sea moss by weight of the composition. In another embodiment, the composition includes 25% to 30% sea moss by weight of the composition.

In an embodiment, the composition includes 30% to 95% sea moss by weight of the composition. In another embodiment, the composition includes 30% to 90% sea moss by weight of the composition. In another embodiment, the composition includes 30% to 85% sea moss by weight of the composition. In another embodiment, the composition includes 30% to 80% sea moss by weight of the composition. In another embodiment, the composition includes 30% to 75% sea moss by weight of the composition. In another embodiment, the composition includes 30% to 70% sea moss by weight of the composition. In another embodiment, the composition includes 30% to 65% sea moss by weight of the composition. In another embodiment, the composition includes 30% to 60% sea moss by weight of the composition. In another embodiment, the composition includes 30% to 55% sea moss by weight of the composition. In another embodiment, the composition includes 30% to 50% sea moss by weight of the composition. In another embodiment, the composition includes 30% to 45% sea moss by weight of the composition. In another embodiment, the composition includes 30% to 40% sea moss by weight of the composition. In another embodiment, the composition includes 30% to 35% sea moss by weight of the composition.

In an embodiment, the composition includes 35% to 95% sea moss by weight of the composition. In another embodiment, the composition includes 35% to 90% sea moss by weight of the composition. In another embodiment, the composition includes 35% to 85% sea moss by weight of the composition. In another embodiment, the composition includes 35% to 80% sea moss by weight of the composition. In another embodiment, the composition includes 35% to 75% sea moss by weight of the composition. In another embodiment, the composition includes 35% to 70% sea moss by weight of the composition. In another embodiment, the composition includes 35% to 65% sea moss by weight of the composition. In another embodiment, the composition includes 35% to 60% sea moss by weight of the composition. In another embodiment, the composition includes 35% to 55% sea moss by weight of the composition. In another embodiment, the composition includes 35% to 50% sea moss by weight of the composition. In another embodiment, the composition includes 35% to 45% sea moss by weight of the composition. In another embodiment, the composition includes 35% to 40% sea moss by weight of the composition.

In an embodiment, the composition includes 40% to 95% sea moss by weight of the composition. In another embodiment, the composition includes 40% to 90% sea moss by weight of the composition. In another embodiment, the composition includes 40% to 85% sea moss by weight of the composition. In another embodiment, the composition includes 40% to 80% sea moss by weight of the composition. In another embodiment, the composition includes 40% to 75% sea moss by weight of the composition. In another embodiment, the composition includes 40% to 70% sea moss by weight of the composition. In another embodiment, the composition includes 40% to 65% sea moss by weight of the composition. In another embodiment, the composition includes 40% to 60% sea moss by weight of the composition. In another embodiment, the composition includes 40% to 55% sea moss by weight of the composition. In another embodiment, the composition includes 40% to 50% sea moss by weight of the composition. In another embodiment, the composition includes 40% to 45% sea moss by weight of the composition.

In an embodiment, the composition includes 45% to 95% sea moss by weight of the composition. In another embodiment, the composition includes 45% to 90% sea moss by weight of the composition. In another embodiment, the composition includes 45% to 85% sea moss by weight of the composition. In another embodiment, the composition includes 45% to 80% sea moss by weight of the composition. In another embodiment, the composition includes 45% to 75% sea moss by weight of the composition. In another embodiment, the composition includes 45% to 70% sea moss by weight of the composition. In another embodiment, the composition includes 45% to 65% sea moss by weight of the composition. In another embodiment, the composition includes 45% to 60% sea moss by weight of the composition. In another embodiment, the composition includes 45% to 55% sea moss by weight of the composition. In another embodiment, the composition includes 45% to 50% sea moss by weight of the composition.

In an embodiment, the composition includes 50% to 95% sea moss by weight of the composition. In another embodiment, the composition includes 50% to 90% sea moss by weight of the composition. In another embodiment, the composition includes 50% to 85% sea moss by weight of the composition. In another embodiment, the composition includes 50% to 80% sea moss by weight of the composition. In another embodiment, the composition includes 50% to 75% sea moss by weight of the composition. In another embodiment, the composition includes 50% to 70% sea moss by weight of the composition. In another embodiment, the composition includes 50% to 65% sea moss by weight of the composition. In another embodiment, the composition includes 50% to 60% sea moss by weight of the composition. In another embodiment, the composition includes 50% to 55% sea moss by weight of the composition.

In an embodiment, the composition includes 55% to 95% sea moss by weight of the composition. In another embodiment, the composition includes 55% to 90% sea moss by weight of the composition. In another embodiment, the composition includes 55% to 85% sea moss by weight of the composition. In another embodiment, the composition includes 55% to 80% sea moss by weight of the composition. In another embodiment, the composition includes 55% to 75% sea moss by weight of the composition. In another embodiment, the composition includes 55% to 70% sea moss by weight of the composition. In another embodiment, the composition includes 55% to 65% sea moss by weight of the composition. In another embodiment, the composition includes 55% to 60% sea moss by weight of the composition.

In an embodiment, the composition includes 60% to 95% sea moss by weight of the composition. In another embodiment, the composition includes 60% to 90% sea moss by weight of the composition. In another embodiment, the composition includes 60% to 85% sea moss by weight of the composition. In another embodiment, the composition includes 60% to 80% sea moss by weight of the composition. In another embodiment, the composition includes 60% to 75% sea moss by weight of the composition. In another embodiment, the composition includes 60% to 70% sea moss by weight of the composition. In another embodiment, the composition includes 60% to 65% sea moss by weight of the composition.

In an embodiment, the composition includes 65% to 95% sea moss by weight of the composition. In another embodiment, the composition includes 65% to 90% sea moss by weight of the composition. In another embodiment, the composition includes 65% to 85% sea moss by weight of the composition. In another embodiment, the composition includes 65% to 80% sea moss by weight of the composition. In another embodiment, the composition includes 65% to 75% sea moss by weight of the composition. In another embodiment, the composition includes 65% to 70% sea moss by weight of the composition.

In an embodiment, the composition includes 70% to 95% sea moss by weight of the composition. In another embodiment, the composition includes 70% to 90% sea moss by weight of the composition. In another embodiment, the composition includes 70% to 85% sea moss by weight of the composition. In another embodiment, the composition includes 70% to 80% sea moss by weight of the composition. In another embodiment, the composition includes 70% to 75% sea moss by weight of the composition.

In an embodiment, the composition includes 75% to 95% sea moss by weight of the composition. In another embodiment, the composition includes 75% to 90% sea moss by weight of the composition. In another embodiment, the composition includes 75% to 85% sea moss by weight of the composition. In another embodiment, the composition includes 75% to 80% sea moss by weight of the composition.

In an embodiment, the composition includes 80% to 95% sea moss by weight of the composition. In another embodiment, the composition includes 80% to 90% sea moss by weight of the composition. In another embodiment, the composition includes 80% to 85% sea moss by weight of the composition.

In an embodiment, the composition includes 85% to 95% sea moss by weight of the composition. In another embodiment, the composition includes 85% to 90% sea moss by weight of the composition.

In an embodiment, the composition includes 90% to 95% sea moss by weight of the composition.

In an embodiment, the composition includes 5% sea moss by weight of the composition. In another embodiment, the composition includes 10% sea moss by weight of the composition. In another embodiment, the composition includes 15% sea moss by weight of the composition. In another embodiment, the composition includes 20% sea moss by weight of the composition. In another embodiment, the composition includes 25% sea moss by weight of the composition. In another embodiment, the composition includes 30% sea moss by weight of the composition. In another embodiment, the composition includes 35% sea moss by weight of the composition. In another embodiment, the composition includes 40% sea moss by weight of the composition. In another embodiment, the composition includes 45% sea moss by weight of the composition. In another embodiment, the composition includes 50% sea moss by weight of the composition. In another embodiment, the composition includes 55% sea moss by weight of the composition. In another embodiment, the composition includes 60% sea moss by weight of the composition. In another embodiment, the composition includes 65% sea moss by weight of the composition. In another embodiment, the composition includes 70% sea moss by weight of the composition. In another embodiment, the composition includes 75% sea moss by weight of the composition. In another embodiment, the composition includes 80% sea moss by weight of the composition. In another embodiment, the composition includes 85% sea moss by weight of the composition. In another embodiment, the composition includes 90% sea moss by weight of the composition. In another embodiment, the composition includes 95% sea moss by weight of the composition.

In an embodiment, a method of making a composition for a beauty product includes the steps of:
Obtaining a quantity of Aloe Vera plant. In an embodiment, the Aloe Vera plant is obtained from a supermarket, farmers market or grocery store.
Obtaining a quantity of sea moss. In an embodiment, the sea moss is obtained from a supermarket, farmers market or grocery store.
Rinsing the sea moss with water for a period of 30 seconds to two minutes. In an embodiment, the rinsing step facilitates the removal of dirt and sand adhered to the sea moss. In an embodiment, the temperature of the water is lukewarm. In an embodiment, the temperature of the water is 95° F. to 115° F.
Storing the sea moss in a refrigeration unit for a period. In an embodiment, the refrigeration unit is a standard household refrigerator. In an embodiment, the sea moss is stored in the refrigeration unit at a temperature of 35° F. to 45° F. In an embodiment, the sea moss is stored in the refrigeration unit for 4 hours to 36 hours. In an embodiment, the sea moss is stored in the refrigeration unit for 24 hours.
Mixing the sea moss. In an embodiment, the mixing step includes mixing the sea moss in a blender to grind down the sea moss. In an embodiment, the blender is a standard household blender. In an embodiment, the weight of the sea moss is 32 g (about 1 ounce). In an embodiment, the weight of the sea moss is 10 grams to 50 grams.
Storing the mixed sea moss in a refrigeration unit for a period. In an embodiment, the refrigeration unit is a standard household refrigerator. In an embodiment, the mixed sea moss is stored in the refrigeration unit at a temperature of 35° F. to 45° F. In an embodiment, the mixed sea moss is stored in the refrigeration unit for a period of 4 hours to 36 hours. In an embodiment, the mixed sea moss is stored in the refrigeration unit for 24 hours.
Cutting the exterior rind/cuticle of the leaf portion of the Aloe vera plant and obtaining an interior gel of the Aloe vera plant. In an embodiment, the weight of the Aloe gel obtained is 32 g (about 1 ounce). In an embodiment, the weight of the Aloe gel obtained is 10 grams to 50 grams.
Mixing the sea moss with the Aloe vera gel. In an embodiment, 1 ounce of the sea moss is mixed with 1 ounce of the Aloe gel. In an embodiment the sea moss and the Aloe vera gel are mixed with one another for 30 seconds to two minutes. In an embodiment the sea moss and the Aloe vera gel are mixed with one another for one minute. In an embodiment, the mixing step includes mixing the sea moss and the Aloe vera gel in a blender. In an embodiment, the blender is a standard household blender. In an embodiment, the mixing step includes mixing the sea moss and the Aloe vera gel manually (e.g., in a bowl, container, receptacle).
Optionally, storing the composition (i.e., mixed sea moss and Aloe vera gel) in a refrigeration unit for a period.

In an embodiment, the composition is used as a concealer primer. In an embodiment, the concealer is configured to cover a person's imperfections underneath their eyes. In an embodiment, the concealer facilitates keeping make up on the skin for an extended period. In an embodiment, the composition reduces puffiness under a person's eyes while filling in fine lines to create an image of beauty and flawless skin.

In an embodiment, the composition includes a topical preparation. In an embodiment, the composition includes a body preparation. In an embodiment, the composition includes a skin preparation. In an embodiment, the composition includes a cream. In another embodiment, the composition includes a lotion.

What is claimed is:

1. A composition, comprising:
Aloe vera gel, wherein the Aloe vera gel is 5% to 95% by weight of the composition; and
sea moss, wherein the sea moss is 5% to 95% by weight of the composition.

2. The composition of claim 1, wherein the sea moss is Chondrus crispus.

3. The composition of claim 1, wherein the composition is configured for us a concealer primer.

4. The composition of claim 1, wherein the composition is configured for use as a topical preparation.

5. The composition of claim 1, wherein the composition is configured for use as a skin preparation.

6. The composition of claim 1, wherein the composition is configured for use as a body preparation.

7. The composition of claim 1, wherein the composition is configured for use as a cream.

8. The composition of claim 1, wherein the composition is configured for use as a lotion.

9. A method, comprising the steps of:
obtaining a quantity of Aloe Vera plant;
obtaining a quantity of sea moss;
rinsing the sea moss with water for a period of 30 seconds to two minutes, wherein a temperature of the water is 95° F. to 115° F.;
storing the sea moss in a refrigeration unit for a period of 4 hours to 36 hours;
mixing the sea moss;
storing the mixed sea moss in a refrigeration unit for a period of 4 hours to 36 hours;
obtaining an interior gel of the Aloe vera plant; and
mixing the sea moss with the Aloe vera gel.

10. The method of claim 9, further comprising the step of storing the mixed sea moss and the Aloe vera gel in a refrigeration unit for a period.

11. The method of claim 9, wherein the sea moss of the storing the sea moss step is stored in the refrigeration unit at a temperature of 35° F. to 45° F.

12. The method of claim 9, wherein the mixed sea moss of the storing the mixed sea moss step is stored in the refrigeration unit at a temperature of 35° F. to 45° F.

13. The method of claim 9, wherein the sea moss of the storing the sea moss step is stored in the refrigeration unit for 24 hours.

14. The method of claim 9, wherein the mixed sea moss of the storing the mixed sea moss step is stored in the refrigeration unit for 24 hours.

15. The method of claim 9, wherein the mixing the sea moss step includes mixing the sea moss in a blender.

16. The method of claim 9, wherein a weight of the sea moss is 10 grams to 50 grams.

17. The method of claim 16, wherein a weight of the Aloe vera gel is 10 grams to 50 grams.

18. The method of claim 17, wherein the mixing the mixed sea moss with the Aloe vera gel step includes mixing the mixed sea moss and the Aloe vera gel for 30 seconds to two minutes.

19. The method of claim 18, wherein the mixing the mixed sea moss with the Aloe vera gel step includes mixing the mixed sea moss and the Aloe vera gel in a blender.

20. The method of claim 18, wherein the mixing the mixed sea moss with the Aloe vera gel step includes mixing the mixed sea moss and the Aloe vera gel manually.
